(19) United States
(12) Patent Application Publication
(10) Pub. No.: US 2024/0032943 A1
(43) Pub. Date: Feb. 1, 2024

(54) VASCULAR BLEEDING CONTROL DEVICE AND METHODS OF USE

(71) Applicant: Medcura, Inc., Riverdale, MD (US)

(72) Inventors: Matthew B. DOWLING, Riverdale, MD (US); David A. PARROTT, Cincinnati, OH (US); Justin MCCARTHY, Boxborough, MA (US); Jesse DRAKE, Westborough, MA (US)

(21) Appl. No.: 18/043,233

(22) PCT Filed: Aug. 28, 2021
(86) PCT No.: PCT/US2021/048119

§ 371 Date: Feb. 27, 2023

Related U.S. Application Data
(60) Provisional application No. 63/072,112, filed on Aug. 29, 2020.

(51) Int. Cl. A61B 17/132; A61B 90/00

(52) U.S. Cl. CPC A61B 17/1325; A61B 90/08; A61B 2017/00407

(57) ABSTRACT

A vascular bleeding control device that is used in managing bleeding at a puncture site, for example, after a surgical procedure when a large needle is required. The device has a resilient lever that extends away from a frame. The resilient lever extends away from the frame and resists pressure applied towards the frame. A pad on an outward face of the resilient lever contacts the puncture site and assists in managing bleeding until the blood vessel is sufficiently healed. The pad is manufactured of a hemostatic material that assists in decreasing healing time and can be easily removed from the puncture site without reopening the wound.

### FIELD OF THE INVENTION

The present application relates generally to devices for controlling vascular bleeding. More particularly, the application relates to an apparatus for controlling bleeding from a punctured vascular vessel by applying pressure through a resilient lever at the bleeding site.

### BACKGROUND OF THE INVENTION

There are many devices used to control bleeding in various parts of an individual's body. Such devices apply pressure at the wound or upstream from the bleeding site. For example, U.S. Pat. No. 8,303,620 describes a torniquet article that has a substantially rigid base, a handle, a strap, and a structural member that allows single-handed application of the torniquet to a bleeding extremity in a patient's body.

United States Application Publication Number 2012/0053617 describes a vascular compression apparatus, pad, and method that is used for applied to a patient's limb and causing partial or full occlusion of a blood vessel. Pads on the device apply preferential compression to portions of a circumference of a limb so as to enable blood flow through adjacent blood vessels during the compression period. The device has a threaded strap that is used to tightened the device around the patient's limb and apply pressure on the pad that, in turn, places pressure on the blood vessel.

A radial compression hemostasis band with doppler confirming vascular patency is described in United States Application Publication Number 2014/0142615. The radial compression band is used to apply radial pressure on the forearm of a patient. The radial compression band has an elongated arm band, an inflatable bladder, a slider, and a pressure pad. Once the band is applied to an extremity, such as a wrist, the pressure against a bleeding wound is adjusted by inflating or deflating the bladder.

An adjustable ratcheting vascular compression device is described in U.S. Pat. No. 9,433,423. The ratcheting adjustable vascular compression device assists in achieving partial or full occlusion of a blood vessel when applied to a patient's limb, during or following a medical procedure. The device has a ratcheting strap and a pad on the side that is to contact the patient's blood vessel. The pad is used to apply pressure on the blood vessel. Pressure is applied by the ratcheting strap. A similar straps are described in U.S. Pat. No. 5,269,803 and Other adjustable compression straps are described in U.S. Pat. Nos. 6,752,820; 8,657,850; and 8,708,991. A compression bandage is described in EP 2 647 359. The compression bandage has a frame and a firm pressure bar that applies pressure to a wound when the strap is tightened.

U.S. Pat. No. 9,433,423 explains that one of the problems encountered during clinical procedures involving radial arterial punctures is that it is necessary to progressively release compression over the vessel to increase blood flow gradually while not disturbing the clot formed during hemostasis. The devices described above achieve these goal in different ways. In one example, an inflatable pad can be used which can be adjusted by increasing or decreasing air pressure. In other embodiments, mechanical adjustments can be achieved using a ratchet strap or having a pad that can be adjusted mechanically. Patent '423 further explains that “continued perfusion through the radial artery during the period in which external compression is applied for the purpose of achieving hemostasis at the arteriotomy and puncture site is required in order to reduce the incidence of chronic radial artery occlusion, a not-uncommon complication of radial access.” Patent hemostasis refers to patency of the radial artery during the hemostasis period. Adjusting compression to permit patent hemostasis helps avoid chronic radial artery occlusion. It is difficult, however, for clinicians to be able to determine when the necessary adjustments can be made and when additional pressure adjustments are needed. Thus, there is a need for an easily adjustable, secure, vascular compression device that also allows clinicians to easily determine if appropriate pressure is being applied.

### SUMMARY OF THE INVENTION

One embodiment of a vascular bleeding control device is disclosed that comprises a frame and a resilient lever, wherein the resilient lever extends away from the frame and resists pressure applied to the resilient lever towards the frame. Further embodiment is disclosed of a vascular bleeding control device that comprises a frame and a resilient lever, wherein the resilient lever extends away from the frame and resists pressure applied to the resilient lever towards the frame; and a strap.

A method for controlling vascular bleeding, is described, in which one step is placing a vascular bleeding control device comprising a frame and a resilient lever, wherein the resilient lever extends away from the frame and resists pressure applied to the resilient lever towards the frame; on a puncture site and adjusting the device to control blood flow and hemostasis.

### BRIEF DESCRIPTION OF THE DRAWINGS

The invention will be better understood with reference to the drawings, wherein:

FIG. 1 is a perspective view of a vascular bleeding control device.

FIG. 2 is a top planar view of the vascular bleeding control device.

FIG. 3 is a front view of the vascular bleeding control device, as seen from the perspective of a ratchet strap fastening end.

FIG. 4 is a left side view of the vascular bleeding control device.

FIG. 5 is a rear view of the vascular bleeding control device, as seen from the perspective of an anchor end.

FIG. 6 is a left side perspective view of the vascular bleeding control device with a ratchet strap.

FIG. 7 is a right side perspective view of the vascular bleeding control device with a ratchet strap.

FIG. 8 is a top view of the vascular bleeding control device with a ratchet strap.

FIG. 9 is a left side view of the vascular bleeding control device with a ratchet strap.

FIG. 10 is a perspective view of the vascular bleeding control device with a ratchet strap secured through a locking end.

FIG. 11 is a side view of the vascular bleeding control device with a ratchet strap secured through a locking end.

### DETAILED DESCRIPTION

As shown in the figures, a vascular bleeding control device 100 is described that aids patients recover from procedures which involve vascular bleeding requiring adjustable pressure to address patent hemostasis. It is to be understood that the embodiments described herein are exemplary in nature and those skilled in the art may modify the described embodiments in order to achieve the objectives of described in the present application.

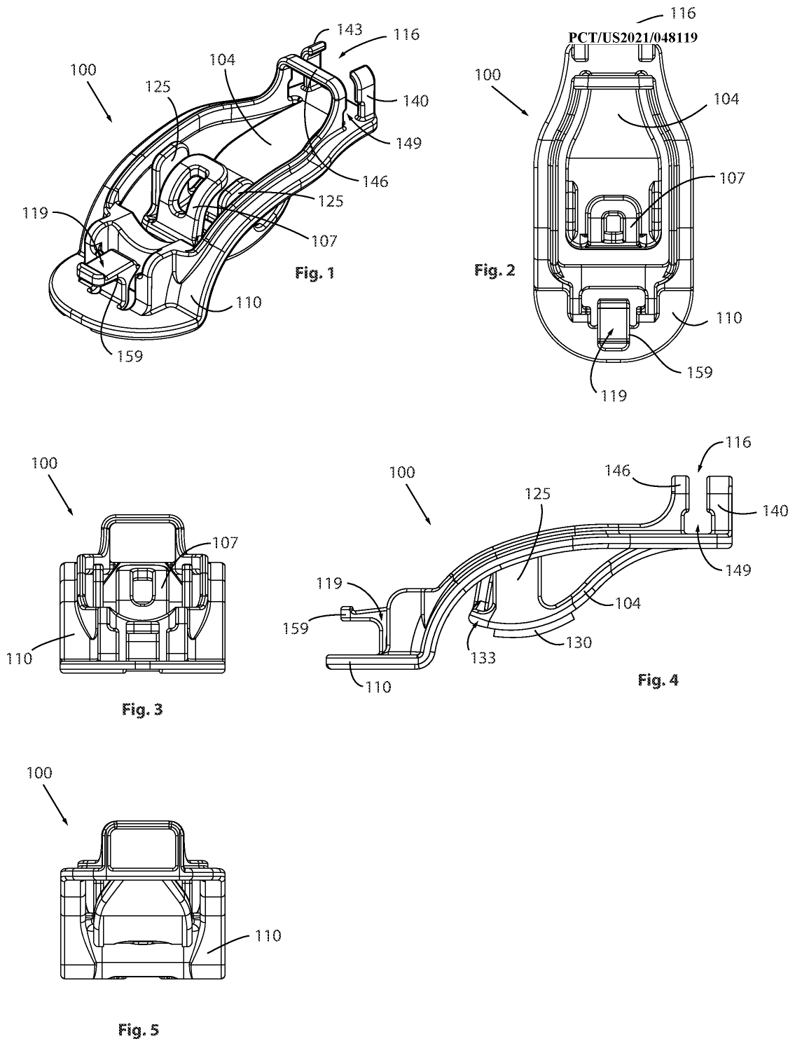

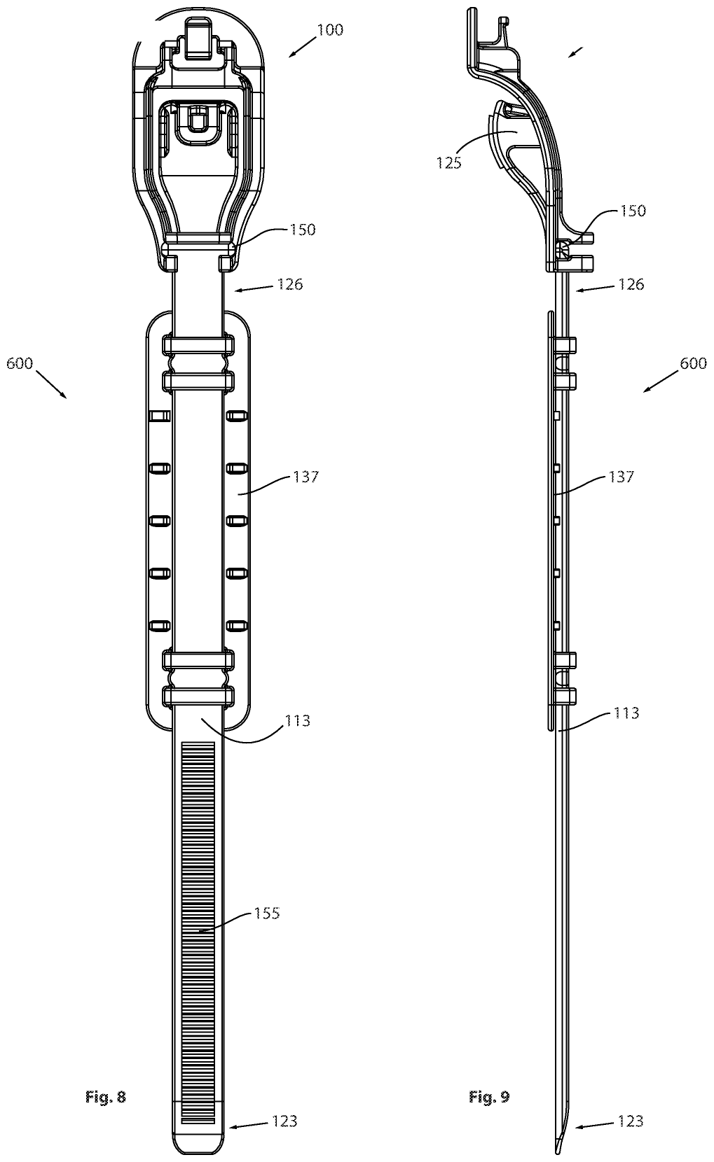

FIGS. 1 through 11 show various views of one embodiment of the vascular bleeding control device 100. In one exemplary embodiment, the device 100 has two basic parts: a frame 110 and a resilient lever 104. The resilient lever 104 extends away from the frame 110 and resists pressure applied to the resilient lever 104 towards the frame 110. As utilized herein, a resilient lever 104 is flexible in that when force is applied to the resilient lever 104, the resilient lever is capable of displacing in a desired direction, in this case towards the frame 110. Once the force is removed, the resilient lever 104 returns to a starting position along a neutral plane. The neutral plane of the vascular bleeding control device 100 is a parallel plane to the long axis of the vascular bleeding control device 100, which neutral plan runs from a securing end 116 to a locking end 119 of the vascular bleeding control device 100. Although the resilient lever 104 can be displaced towards the frame 110, the resilient lever 104 continues to oppose the force applied towards the frame 110. When used to treat a punctured blood vessel, the resilient lever 104 applies pressure to the punctured site. It is contemplated that the device 100 may be utilized in any wound that requires radial pressure. Typically, such wounds are found in the extremities of patients, such as wrists, arms, ankles, and legs. In one embodiment, the device is utilized in wrist wounds that result from cannulas being used during medical or surgical procedures.

The device 100 is constructed of resilient, flexible, elastic material, such as a plastic, rubber, silicone, thermoplastics, polymeric materials, or urethane materials. In some preferred embodiments the resilient material is polycarbonate. The resilient material is one that allows the resilient lever 104 to be pulled towards the frame 110 on a vertical axis, while maintaining force away from the frame 110. The vertical axis of the device is perpendicular to a plane that runs along the length and width the device 100.

The resilient lever 104 has a moment of inertia of between 1.5×10⁻⁵ in⁴ and 9.0×10⁻⁵ in⁴ for a standard exemplary embodiment of the vascular bleeding control device 100. The standard vascular bleeding control device 100 is used, typically, when small punctures are involved. The resilient lever 104 in a second exemplary embodiment has a moment of inertia of between 5.0×10⁻⁵ in⁴ and 2.0×10⁻⁴ in⁴ for bariatric applications where stronger downward force is required. In a third embodiment, the resilient lever 104 has a moment of inertia of between 1.0×10⁻⁵ in⁴ and 4.0×10⁻⁵ in⁴ for pediatric applications. The moment of inertia of a rectangular cross-section can be calculated as follows: multiplying the base length (parallel to the neutral plane) by the cube of the height length (perpendicular to the neutral plane) divided by 12. For other lever cross-sections, similar formulas can be used and are familiar to those skilled in the art. The moment of inertia determines how much force the resilient lever 104 applies to a wound or puncture site.

In some embodiments, the device 100 also includes a depressor 107 that allows the resilient lever to be pulled towards the frame 110. The depressor 107, in one embodiment, is shaped as a semi-circle towards a securing end 116 of the device 100. The depressor 107, allows an individual to pull the resilient lever 104 towards the frame 110 with a single finger. A person of ordinary skill in the art would recognize that the depressor 107 can be shaped in any appropriate shape that allows the user to pull the depressor 107 towards the frame 110. The ability to pull the depressor 107 towards the frame, allows a the patient or healthcare provider to check the condition of the wound easily. The ability to pull the depressor 107 away from the wound also allows the device to be adjusted to ensure proper blood flow is maintained, while allowing clotting and healing to occur.

The resilient lever 104 also includes one or more position indicators 125 that allow the user to determine what type of adjustment is necessary for the device 100. The position indicators 125, in some embodiments, are posts that may be printed, colored or etched to communicate the force applied by the resilient lever 104, based on the displacement of the lever relative to the frame 110. As the device 100 is tightened the position indicator 125 rises past the edge of the frame 110. The user may adjust the device to a preselected indication that corresponds to the force that the resilient lever 104 applies to the puncture site.

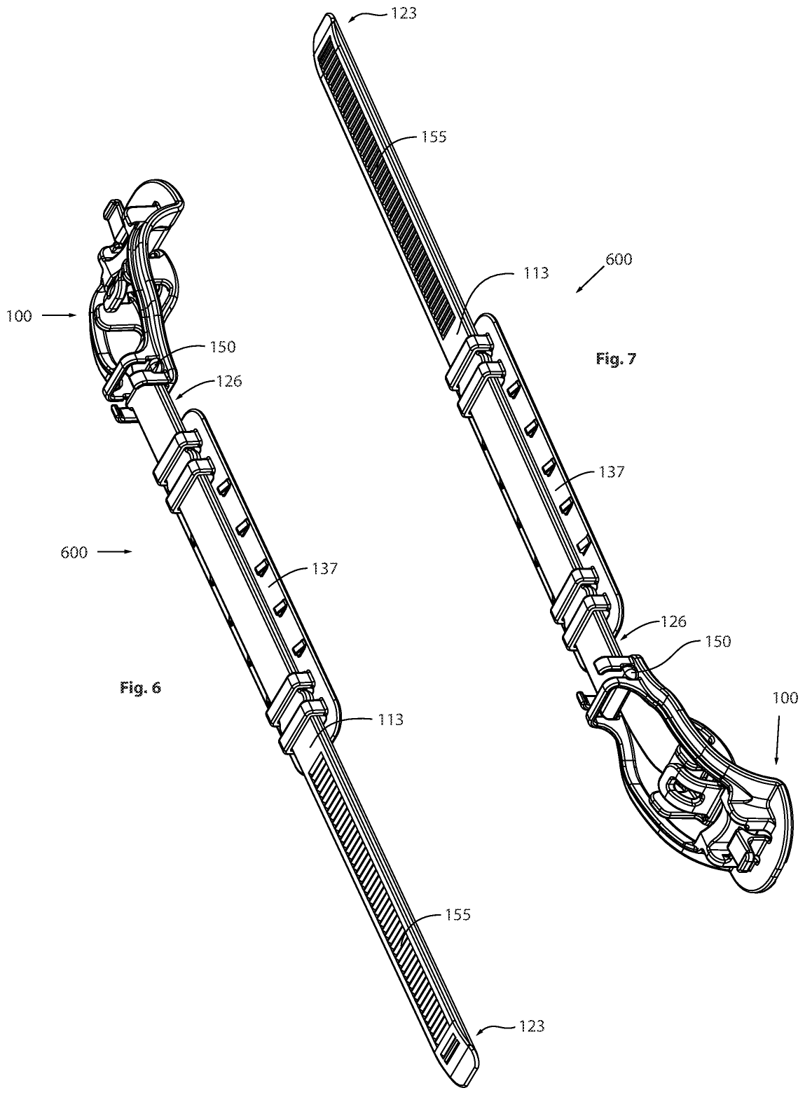

As shown in FIGS. 6 through 10 the vascular bleeding control device 100 further comprises a strap 113. The combination of the vascular bleeding control device 100 and a strap 113 form a single apparatus 600. The strap 113, or fastening component, is designed to keep the vascular bleeding control device 100 in place at a puncture or wound site and to ensure appropriate pressure is being placed on the bleeding vessel. The strap 113 or fastening device, in some instances is a hoop and loop fastener strap. In other embodiments, the strap 113 is a ratchet fastener, a zipper type fastener, or a hoop and loop fastener. A ratchet type strap is one that includes a plurality of groves 155 for locking the strap 113 in place. The strap 113 is manufactured from various materials. In some embodiments, the strap 113 is made of various resilient but flexible materials such as silicone, thermoplastic, thermoplastic elastomer or thermoplastic urethane. In some embodiments, the strap 113 includes a safety feature that allows the strap to stretch when a specified tension is placed on the strap 113. This feature prevents applying too much pressure at the site where vascular bleeding control device 100 is placed.

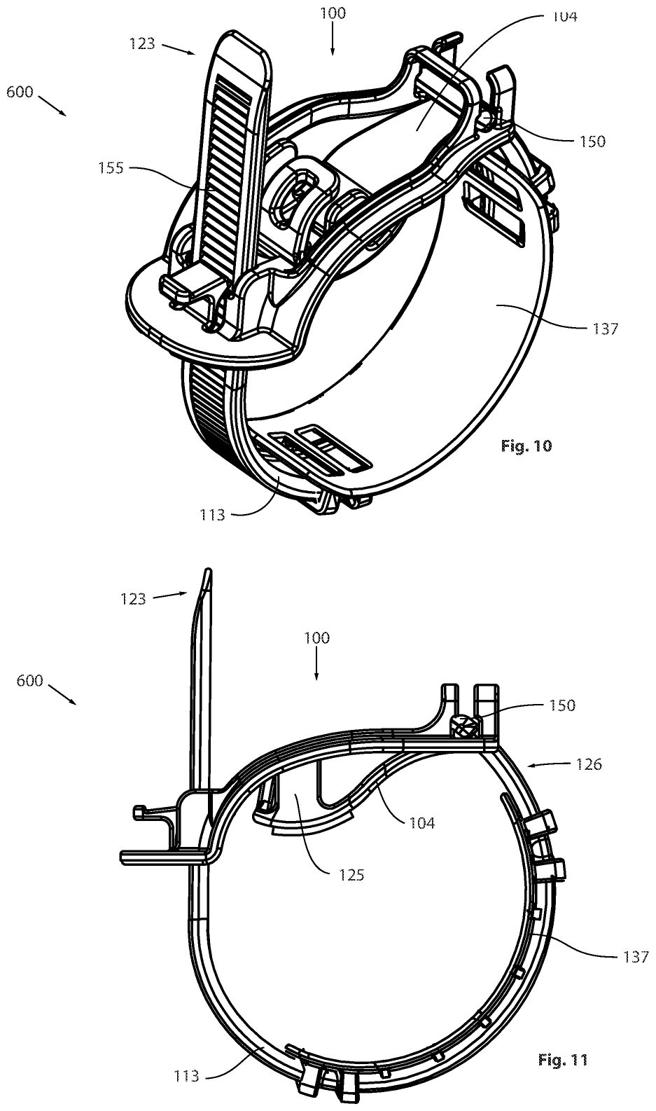

As shown in FIGS. 10 and 11, the vascular bleeding control device 100 further comprises a securing end 116 and a locking end 119. The strap 113 is secured to the vascular bleeding control device 100 at the securing end 116. In some embodiments, the securing end 116 has a first rear post 140 and a second rear post 143. The securing end 116 further includes a guard 146. The first rear post 140 is opposite on one side to the second rear post 143 and on a second side perpendicular to the first side to the guard 146. The second rear post 143 is opposite on one side to the first rear post 140 and on a second side perpendicular to the first side to the guard 146, where the first rear post 140, the second rear post 143 and the guard 146 form a channel 149.

The strap 113 comprises an anchor end 126 and a fastening end 123. The anchor end 126 connects to the securing end 116 of the vascular bleeding control device 100. In some embodiments, the anchor end 126 is just an extension of the anchor end 126, which means that the anchor end 126 is made of a similar material than the securing end 116 or of a material that can be fused together with the securing end 116. In other embodiments, the anchor end 126 of the strap 113 is glued otherwise fixed to the securing end 116 of the vascular bleeding control device 100. In yet further embodiments, such as shown in FIGS. 6 and 7, the anchor end 126 has a locking bar 150 or other similar mechanism that fits in channel 149 and is locked in place by locking recesses on the first rear post 140, second rear post 143, and the guard 146. The locking recesses, in some embodiments, are cut outs in the first rear post 140, second rear post 143, and the guard 146, that prevent the strap 113 from separating from the vascular bleeding control device 100 once put in place. In some embodiments, protrusions on the sides of the strap 113 fit in the channel 149 and locks the strap 113 in place.

In some embodiments, the vascular bleeding control device 100 also includes a slider 137. The slider 137 is wider than the strap 113 and protects a patient's skin around the area on which the device 100 is utilized. For example, when the strap 113 is narrow, it may cut into the skin of the patient's wrist. In such cases, the device 113 is placed around a wrist, the slider 137 protects the skin around the wrist from being cut by the narrower strap 113. The slider 137, in various embodiments, is made of many different materials that provide protection to the skin surrounding the wound.

The fastening end 123 of the strap 113, in some embodiments where a ratcheting strap is utilized, fits the locking end 119. The strap includes a plurality of groves 155, which catch within a mechanism in the locking end 119. The locking end 119, has a release lever 159. The release lever 159 is resilient and maintains pressure on the strap 113 unless pulled away by a user. The groves 155 catch on the release lever 159. In order to change the diameter of the apparatus 600, i.e., the combination of the device 100 and the strap 113, the release lever 159 is depressed away from the groves 155. When the release lever 159 is depressed away from the groves 155, the strap 113 can slide freely through the locking end 119 of the device 100. Once pressure from the release lever 159 is removed, the release lever 159 puts pressure on the groves 155 preventing the strap 113 from sliding through the locking end 119 of the device 100. Utilizing this mechanism, the vascular bleeding control device 100 is adjusted based on the indications found on the position indicators 125 as described above. When the vascular bleeding control device 100 and strap 113 are used to manage vascular bleeding on a patient, the use of the release lever 159 results in a decrease on the pressure applied to the puncture wound by the device 100. In other embodiments, a strap 113—that is not a ratcheting strap but a hook and loop type fastener—is secured at the locking end 119 by a bar. The strap 113 passes through one side of the bar, loops around the bar, and secures onto itself on the other side of the strap 113. Utilizing this mechanism, the vascular bleeding control device 100 is adjusted based on the indications found on the position indicators 125 as described above.

In some embodiments, the strap 113 has a width of ½″-¾″. The strap 113, in some embodiments, has a thickness of 0.07″-0.125″. The slider 137, in some embodiments has a width of 1″-1.5″. The width of the slider 137 has a width that prevents the strap from injuring a patient's skin.

The resilient lever 104 of the device 100 has an outward face 133. The outward face 133 is the portion of the resilient lever 104 that faces away from the frame 110 and comes in contact with a wound when the vascular bleeding control device 100 is used. In some embodiments, the resilient lever 104 has a pad 130 on the outward face 133. The pad 130 is manufactured from a hemostatic material. The hemostatic material, in some embodiments, includes a hydrophobically modified biopolymer. In some embodiments, the pad 130 made entirely of a hydrophobically modified biopolymer. In other embodiments, the pad 130 is made of combinations of materials including the modified biopolymer. The hydrophobically modified biopolymer in some embodiments is in the form of a putty. In further embodiments, the pad is made of gauss material coated with a hydrophobically modified biopolymer.

The hydrophobically modified biopolymer, in some preferred embodiments, is a hydrophobically modified chitosan or alginate. In one embodiment, the hydrophobically modified biopolymer composition has a backbone that have hydrophobic groups attached. In another embodiment, the hydrophobic groups have at least two different sizes. In some embodiments, the hydrophobically modified biopolymer has from about 10% to 50% of available functional groups occupied by a hydrophobic group. In other embodiments, the hydrophobically modified biopolymer has about 5 to 100 moles of hydrophobic groups per mole of polymer. In some embodiments, the biopolymer has from 2 to about 10 different hydrophobic groups, and optionally from 2 to about 5 different hydrophobic groups.

In some embodiments the hydrophobic groups are independently selected from linear, branched, or cyclic hydrocarbon groups. The hydrophobic groups include at least one saturated hydrocarbon, which is optionally a conjugated fatty acid. They hydrophobic modifications, in some embodiments have from 1 to 5 hydrocarbon groups (C1 to C5). In other embodiments, the hydrophobic modifications have 6 to 12 hydrocarbon groups (C6-12). In yet other embodiments, the hydrophobic modifications have 13 to 28 hydrocarbon groups (C13-C28). In further embodiments, the hydrophobic groups have a size greater than 28 hydrocarbon groups (C28). The hydrocarbon modifications may include combinations of the hydrocarbon groups discussed above. In some embodiments the hydrocarbon modifications comprise a C1 to C4 hydrocarbon and a hydrocarbon greater than C6. The C1 to C4 hydrocarbon group is present at 5:1 to 25:1 with respect to hydrocarbon groups of C6 or greater, in some embodiments. In other embodiments, the pad 130 has hydrocarbon groups of from C6 to C12 and a hydrocarbon group of from C16 or C18. In other embodiments, the hydrophobic groups comprise a C1 to C4 hydrocarbon, a C6 to C12 hydrocarbon, and a C16 to C28 hydrocarbon. The modified biopolymer, in some embodiments, is present at 0.1 to about 5% by weight.

In some embodiments, the modified biopolymer further includes an adhesion prevention additive, which facilitates the removal of the pad 130 from the puncture site without removing the clot or and reopening the wound. In one embodiment, the adhesion prevention additive is dextran. The pad 130 has 1.8% wt hydrophobically-modified biopolymer and 0.5% wt dextran particles. In another example, the pad 130 has 1.8% wt hydrophobically-modified biopolymer and 0.6% wt dextran particles. In yet a further embodiment, has 1.8% wt hydrophobically-modified biopolymer and 0.7% wt dextran particles. In other embodiments, the pad 130 has a concentration of 0.5% to 1.0% wt dextran particles.

The vascular bleeding control device 100 and strap 113 described in this application is used in a method for controlling vascular bleeding, particularly after a medical procedure. The method comprises the steps of placing vascular bleeding control device 100 comprising a frame and a resilient lever, wherein the resilient lever 104 extends away from the frame 110 and resists pressure applied to the resilient lever 104 towards the frame, on a puncture site and adjusting the vascular bleeding control device 100 to control blood flow. In some embodiments, one additional step comprises tightening the vascular bleeding control device 100 to increase pressure on the puncture site. A further step comprises loosening the vascular bleeding control device 100 reducing pressure on the puncture site.

In one exemplary embodiment of the method, a patient has a canula at a puncture site. Before removing the cannula, the device 100 and strap 113 are placed on the patient to assist in controlling bleeding after removing the cannula. The strap 113 is tightened around by pulling the fastening end 123 through the locking end 119 of the device 100. The strap 113 is set to a predetermined setting that applies pressure at the puncture site from the resilient lever 104. In a second step, the cannula is removed from the patient. The strap 113 can be adjusted utilizing the release lever 159. The strap 113 is adjusted until the appropriate mark on the position indicator 125 is reached. The Once the cannula is removed and the vascular bleeding control device 100 is in place, the state of the puncture site can be assessed by pulling on the depressor 107. The strap 113, can then be adjusted as necessary to maintain or reduce pressure on the puncture site. The strap vascular bleeding control device 100 can be removed from the patient by releasing the strap 113 once the bleeding as stopped and circulation is determined to be normal.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

What is claimed is:

1. A vascular bleeding control device, comprising:
a frame and a resilient lever, wherein the resilient lever extends away from the frame and resists pressure applied to the resilient lever towards the frame.

2. The vascular bleeding control device of claim 1, further comprising a depressor that allows the resilient lever to be pulled towards the frame.

3. The vascular bleeding control device of claim 1, wherein the resilient lever has a moment of inertia of between 1.5×10⁻⁵ in⁴ and 9.0×10⁻⁵ in⁴; of between 5.0×10⁻⁵ in⁴ and 2.0×10⁻⁴ in⁴; and between 1.0×10⁻⁵ in⁴ and 4.0×10⁻⁵ in⁴.

4. The vascular bleeding control device of claim 1, further comprising a strap.

5. The vascular bleeding control device of claim 4, wherein the strap is a ratchet fastener, zipper fastener, or a hoop and loop fastener.

6. The vascular bleeding control device of claim 4, wherein the strap further comprises an anchor end and a fastening end.

7. The vascular bleeding control device of claim 1, wherein the frame further comprises a locking end and a securing end.

8. The vascular bleeding control device of claim 1, further comprising a pad on an outward face of the resilient lever.

9. The vascular bleeding control device of claim 8, wherein the pad is made from a hemostatic material.

10. The vascular bleeding control device of claim 9, wherein the hemostatic material comprises a hydrophobically modified biopolymer.

11. The vascular bleeding control device of claim 10, wherein the hydrophobically modified biopolymer is a hydrophobically modified chitosan or alginate.

12. The vascular bleeding control device of claim 10, wherein the hydrophobically modified chitosan or alginate comprise hydrocarbon groups selected from linear, branched, or cyclic hydrocarbon groups.

13. The vascular bleeding control device of claim 12, wherein the hydrophobic groups comprise at least one saturated hydrocarbon or a conjugated fatty acid.

14. The vascular bleeding control device of claim 12, wherein hydrophobic groups comprise from 1 to 5 hydrocarbon groups (C1 to C5); 6 to 12 hydrocarbon groups (C6-12); 13 to 28 hydrocarbon groups (C13-C28); or more than 28 hydrocarbon groups (C28).

15. The vascular bleeding control device of claim 12, wherein the hydrocarbon groups comprise combinations of from 1 to 5 hydrocarbon groups (C1 to C5); 6 to 12 hydrocarbon groups (C6-12); 13 to 28 hydrocarbon groups (C13-C28); or more than 28 hydrocarbon groups (C28).

16. The vascular bleeding control device of claim 12; wherein the hydrocarbon groups comprise a C1 to C4 hydrocarbon and a hydrocarbon greater than C6 at 5:1 to 25:1 with respect to hydrocarbon groups of C6 or greater; from C6 to C12 and a hydrocarbon group of from C16 or C18; a C1 to C4 hydrocarbon, a C6 to C12 hydrocarbon, and a C16 to C28 hydrocarbon.

17. The vascular bleeding control device of claim 9, wherein the hemostatic material is a putty.

18. The vascular bleeding control device of claim 4, further comprising a slider.

19. The vascular bleeding control device of claim 18, wherein the slider has a width that prevents the strap from injuring a patient's skin.

20. A vascular bleeding control device, comprising:
a frame and a resilient lever, wherein the resilient lever extends away from the frame and resists pressure applied to the resilient lever towards the frame; and
a strap.

21. The vascular bleeding control device of claim 20, further comprising a depressor that allows the resilient lever to be pulled towards the frame.

22. The vascular bleeding control device of claim 20, wherein the resilient lever has a moment of inertia of between 1.5×10⁻⁵ in⁴ and 9.0×10⁻⁵ in⁴ (standard); of between 5.0×10⁻⁵ in⁴ and 2.0×10′ in⁴ (bariatric); and between 1.0×10⁻⁵ in⁴ and 4.0×10⁻⁵ in⁴ (pediatric).

23. The vascular bleeding control device of claim 20, wherein the strap is a ratchet fastener, zipper fastener, or a hoop and loop fastener.

24. The vascular bleeding control device of claim 20, wherein the strap comprises an anchor end and a fastening end.

25. The vascular bleeding control device of claim 20, wherein the frame further comprises a locking end and a securing end.

26. The vascular bleeding control device of claim 20, further comprising a pad on an outward face of the resilient lever.

27. The vascular bleeding control device of claim 26, wherein the pad is comprises a hemostatic material.

28. The vascular bleeding control device of claim 27, wherein the hemostatic material comprises a hydrophobically modified biopolymer.

29. The vascular bleeding control device of claim 28, wherein the hydrophobically modified biopolymer is a hydrophobically modified chitosan or alginate.

30. The vascular bleeding control device of claim 29, wherein the hemostatic material is a putty.

31. The vascular bleeding control device of claim 20, further comprising a slider.

32. The vascular bleeding control device of claim 31, wherein the slider has a width that prevents the strap from injuring a patient's skin.

33. A method for controlling vascular bleeding, comprising:
placing a vascular bleeding control device comprising a frame and a resilient lever, wherein the resilient lever extends away from the frame and resists pressure applied to the resilient lever towards the frame; on a puncture site and adjusting the device to control blood flow.

34. The method of claim 33, further comprises tightening the vascular bleeding control device.

35. The method of claim 34, further comprising loosening the vascular bleeding control device.

36. The method of claim 34, wherein the vascular bleeding control device is adjusted to an indication on a position indicator of the vascular bleeding control device.

37. The method of claim 36, where in the indication on the position indicator corresponds to a force that the resilient lever applies to the puncture site.

38. A method for controlling vascular bleeding, comprising:
placing a vascular bleeding control device, comprising a frame and a resilient lever, wherein the resilient lever extends away from the frame wherein the resilient lever resists pressure applied to the resilient lever towards the frame; and
a strap on a puncture site; and
adjusting the device to control blood flow.
